(19) United States
(12) Patent Application Publication
(10) Pub. No.: US 2010/0218764 A1
(43) Pub. Date: Sep. 2, 2010

(54) STORAGE SYSTEM FOR AN APPARATUS THAT DELIVERS BREATHABLE GAS TO A PATIENT

(75) Inventors: Philip Rodney KWOK, Chatswood (AU); Perry David Lithgow, Moruya (AU)

Correspondence Address: NIXON & VANDERHYE, PC, 901 NORTH GLEBE ROAD, 11TH FLOOR, ARLINGTON, VA, 22203, US

(73) Assignee: ResMed Limited, Bella Vista (AU)

(21) Appl. No.: 12/770,935

(22) Filed: Apr. 30, 2010

Related U.S. Application Data
(62) Division of application No. 11/483,564, filed on Jul. 11, 2006.
(60) Provisional application No. 60/703,865, filed on Aug. 1, 2005.

(51) Int. Cl. A61M 16/00; A62B 7/02

(52) U.S. Cl. 128/204.18

(57) ABSTRACT

A flow generator assembly includes a flow generator structured to generate a supply of pressurized breathable air to a patient; and a bracket attachable to the flow generator and adapted to mount the flow generator to a bed or a wall adjacent the bed.

### CROSS-REFERENCE TO RELATED APPLICATION

This application is a divisional of 11/483,564, filed Jul. 11, 2006, which claims the benefit of U.S. Provisional Application No. 60/703,865, filed Aug. 1, 2005, each of which is incorporated herein by reference in its entirety.

### FIELD OF THE INVENTION

The present invention relates to an apparatus that delivers breathable gas to a patient.

### BACKGROUND OF THE INVENTION

Apparatus to deliver breathable gas to a patient typically includes multiple components such as a flow generator, an air delivery conduit, and a patient interface. In use, the air delivery conduit delivers pressurized air from the flow generator to the patient interface in contact with the patient's face. The quantity, size, and/or shape of the various components of the apparatus often make it inconvenient or difficult to transport the apparatus from one location to the next. Also, the quantity, size, and/or shape of the various components of the apparatus often make it difficult to store the apparatus in a location that is relatively concealed or unobtrusive and convenient for use. These challenges may adversely affect a patient's decision to use the apparatus, thereby diminishing the effectiveness of therapy.

### SUMMARY OF THE INVENTION

One aspect of the invention is directed towards a storage system for an apparatus to deliver breathable gas to a patient that facilitates the transport and/or convenient storage of the apparatus.

Another aspect of the invention relates to a storage system for an apparatus that delivers a supply of pressurized breathable air to a patient. The apparatus includes a flow generator having at least one of a control panel, a power inlet attachable to a power cord, and an outlet attachable to an air delivery conduit. The storage system includes a container including a first portion and a second portion. The first portion provides a storage compartment adapted to receive the flow generator. The second portion is movably mounted to the first portion for movement between (1) an open position in which the second portion uncovers the storage compartment to allow access to the storage compartment and (2) a closed position in which the second portion closes the storage compartment to inhibit access to the storage compartment. At least one of the first and second portions provides at least one opening therethrough that allows access to at least one of the control panel, the power inlet, and the outlet of the flow generator supported by the container when the container is in the closed position.

Another aspect of the invention relates to a storage system for an apparatus that delivers a supply of pressurized breathable air to a patient. The apparatus includes a flow generator having at least one of a control panel, a power inlet attachable to a power cord, and an outlet attachable to an air delivery conduit. The storage system includes a container including a bottom wall and side walls extending from the bottom wall that define a storage compartment having an upwardly facing opening adapted to receive the flow generator. At least one of the side walls provides at least one opening therethrough to allow access to at least one of the control panel, the power inlet, and the outlet of the flow generator supported by the container.

Yet another aspect of the invention relates to a stand for supporting a flow generator structured to generate a supply of pressurized breathable air to a patient. The stand includes a base and spaced apart arms coupled to the base. The spaced apart arms are configured and arranged to engage opposing walls of the flow generator to stably support the flow generator in a generally vertical orientation.

Still another aspect of the invention relates to a flow generator assembly including a flow generator structured to generate a supply of pressurized breathable air to a patient, and a bracket attachable to the flow generator and adapted to mount the flow generator to a bed or a wall adjacent the bed.

Other aspects, features, and advantages of this invention will become apparent from the following detailed description when taken in conjunction with the accompanying drawings, which are a part of this disclosure and which illustrate, by way of example, principles of the invention.

### BRIEF DESCRIPTION OF THE DRAWINGS

The accompanying drawings facilitate an understanding of the various embodiments of this invention. In such drawings:

FIG. 1 is a perspective view of a storage system for an apparatus to deliver breathable gas constructed according to an embodiment of the present invention, in which the storage system assumes an open position to receive a flow generator and a patient interface of the apparatus;

FIG. 2 is a perspective view of the storage system shown in FIG. 1, in which the storage system is in an open position with the flow generator and patient interface received therein;

FIG. 3 is a perspective view of the storage system shown in FIG. 1, in which the storage system is in a closed position;

FIGS. 3B and 3C illustrate an embodiment of a collapsible air delivery conduit;

FIG. 4 is a perspective view of a storage system for an apparatus to deliver breathable gas constructed according to another embodiment of the present invention;

FIG. 5 is a perspective view of the storage system shown in FIG. 4, in which the storage system is received within a briefcase;

FIG. 6 is a perspective view of the storage system shown in FIG. 4, in which the storage system is received within a cabinet drawer;

FIG. 6B is a perspective view of a storage system according to another embodiment of the present invention;

FIG. 7 is a perspective view of a flow generator of an apparatus to deliver breathable gas constructed in accordance with another embodiment of the present invention, the flow generator being positioned on a table in a horizontal position;

FIGS. 7B and 7C illustrate embodiments of a flow generator including flat motors;

FIG. 8 is a perspective view of the flow generator shown in FIG. 7, the flow generator being positioned beside a bed in an upright position;

FIG. 9 is a perspective view of the flow generator shown in FIG. 7, the flow generator being positioned beneath a mattress of a bed;

FIG. 10 is a perspective view of the flow generator shown in FIG. 7, the flow generator being positioned behind a bed;

FIG. 10B illustrates an embodiment of a bracket for attaching a flow generator behind a bed;

FIG. 11 is a perspective view of a storage system for an apparatus to deliver breathable gas constructed according to still another embodiment of the present invention;

FIG. 12 is a side view of the storage system shown in FIG. 11;

FIG. 13 is a front view of the storage system shown in FIG. 11;

FIG. 14 is a side view of a storage system according to still another embodiment of the present invention; and

FIG. 15 is a front view of the storage system shown in FIG. 14.

### DETAILED DESCRIPTION OF ILLUSTRATED EMBODIMENTS

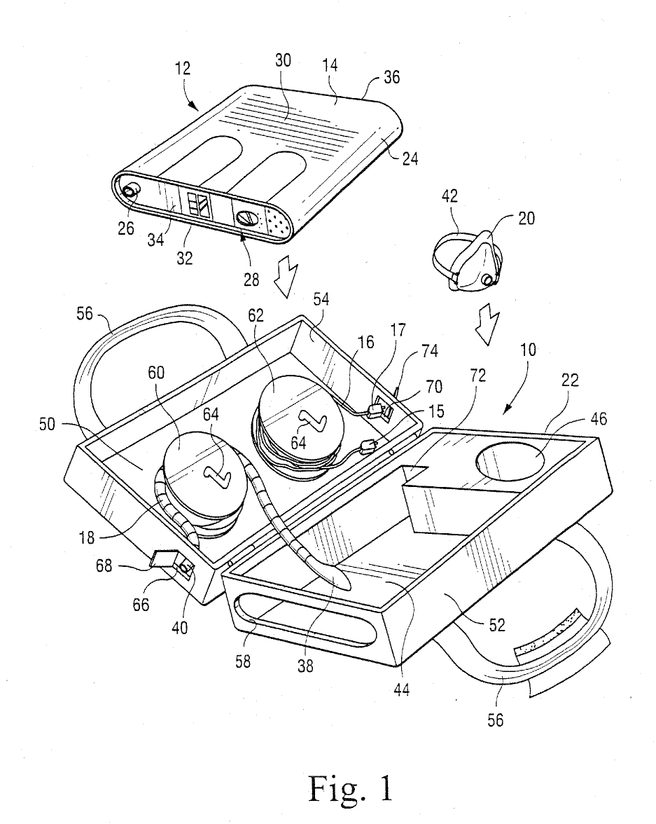

FIGS. 1-3 illustrate a storage system 10 constructed according to an embodiment of the present invention. The storage system 10 is structured for use with an apparatus 12 that delivers a supply of pressurized breathable air to a patient for treatment, e.g., of Sleep Disordered Breathing (SDB) with CPAP or Non-Invasive Positive Pressure Ventilation (NIPPV). The apparatus 12 generally includes a flow generator 14, a power cord 16 for the flow generator 14, an air delivery conduit 18, and a patient interface 20. As discussed in greater detail below, the storage system 10 includes a container 22 adapted to conveniently store the components of the apparatus 12. Moreover, the container 22 is structured to enable operation of the flow generator 14 while it is stored within the container 22.

Flow Generator

The flow generator 14 is structured to generate a supply of pressurized air to be provided to a patient for treatment. The flow generator 14 includes a housing 24 and a blower supported within the housing 24. As is known in the art, the blower is operable to draw a supply of air into the housing 24 through one or more intake openings and provide a pressurized flow of air at an outlet 26.

The flow generator 14 includes a power inlet (not shown) to which a power cord 16 may be selectively engaged for supplying power to the flow generator 14. The power inlet and the power cord 16 may have any suitable structure for supplying power to the flow generator 14. However, the flow generator 14 may be powered in any other suitable manner, e.g., by battery.

The flow generator 14 also includes a control panel 28 that is operable to receive input and to control operation of the flow generator 14 based on the input. The control panel 28 may provide a display screen and one or more control features, e.g., rotatable knob, buttons. The control features may be manually selected to adjust the operating parameters of the flow generator 14.

The housing 24 of the flow generator 14 includes an upper wall 30, a lower wall 32, and front and rear side walls 34, 36. In the illustrated embodiment, the control panel 28 and the outlet 26 of the flow generator 14 are both provided on the front side wall 34 of the housing 24, and the power inlet is provided on the rear side wall 36 of the housing 24. However, the control panel 28, outlet 26, and power inlet may be provided on any suitable wall of the housing 24 and in any suitable arrangement, e.g., common walls, different walls, etc.

Air Delivery Conduit

The supply of pressurized air is delivered to the patient via the air delivery conduit 18 that includes one end 38 coupled to the outlet 26 of the flow generator 14 and an opposite end 40 coupled to the patient interface 20. The air delivery conduit 18 may have any suitable construction, and may be coupled to the flow generator 14 and the patient interface 20 in any suitable manner as is known in the art.

Patient Interface

The patient interface 20 comfortably engages the patient's face and provides a seal. The patient interface 20 may have any suitable configuration as is known in the art, e.g., full-face mask, nasal mask, oro-nasal mask, mouth mask, nasal prongs, etc. Also, any suitable headgear arrangement 42 may be utilized to comfortably support the patient interface 20 in a desired position on the patient's face.

Storage Container

The container 22 of the storage system 10 is in the form of a storage case that provides storage compartments 44, 46 adapted to conveniently store the flow generator 14 and the patient interface 20. Additionally, the storage case 22 provides a cable management system 50 structured to conveniently handle the power cord 16 and the air delivery conduit 18, as discussed in greater detail below.

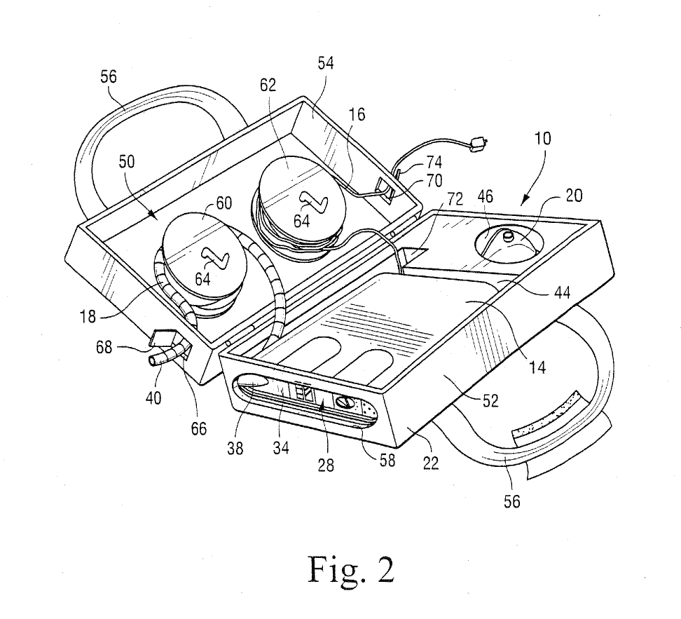

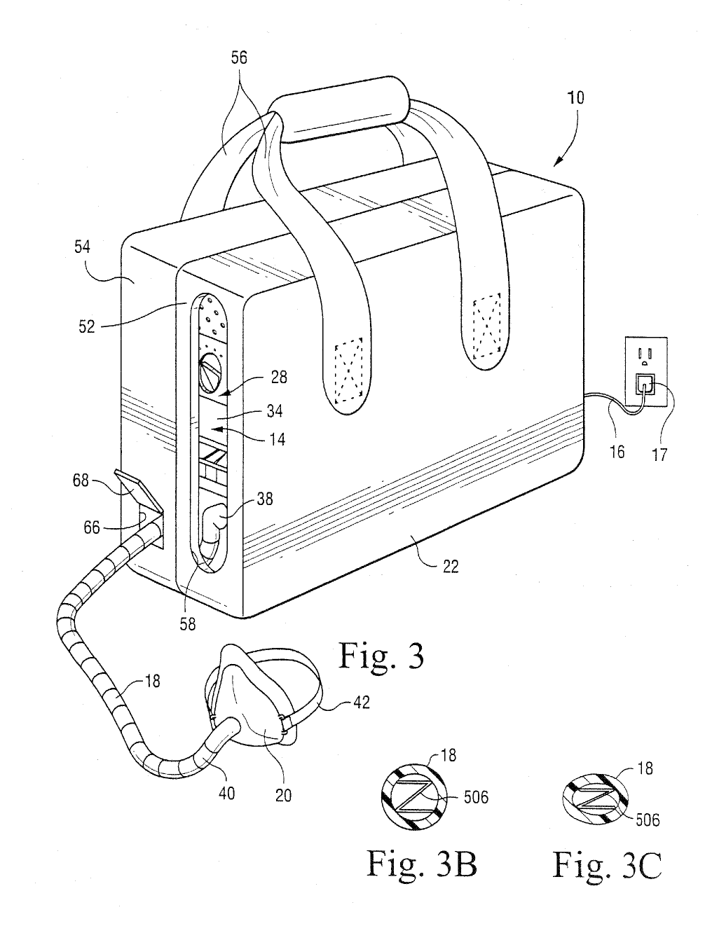

The case 22 includes a first portion 52 and a second portion 54 that are structured to support various components. The first and second portions 52, 54 may include padded or impact resistant walls or surfaces to protect the components. In the illustrated embodiment, the first portion 52 provides the storage compartments 44, 46. The second portion 54 is movably mounted to the first portion 52, e.g., by hinges, for movement between an open position (as shown in FIGS. 1 and 2) and a closed position (as shown in FIG. 3). In the open position, the second portion 54 uncovers the storage compartments 44, 46 to allow access to the storage compartments 44, 46. In the closed position, the second portion 54 closes the storage compartments 44, 46 to inhibit access to the storage compartment 44, 46. Thus, the flow generator 14 and patient interface 20 may be inserted into and removed from respective storage compartments 44, 46 when the case 22 is in the open position, and the case 22 encloses the flow generator 14 and the patient interface 20 to prevent removal therefrom when the case 22 is in the closed position. The case 22 also provides handles 56 that allow one to conveniently carry and transport the case 22 when in the closed position. Additionally, when the case 22 is in the closed position, the first and second portions 52, 54 provide a base structured to maintain the case 22 in a substantially upright position on a support surface as shown in FIG. 3.

In an embodiment, the case 22 may be provided with a mobile mount, e.g., case 22 attaches to a wheel chair to allow patient mobility and equipment protection. Also, the mobile mount may assist to contain noise emissions.

As illustrated, the storage compartments 44, 46 are provided in the first portion 52 of the case 22. The shape and size of each storage compartment 44, 46 generally corresponds with the shape and size of the component to be received therein. For example, the compartment 44 is suitably sized and shaped to receive the flow generator 14, and the compartment 46 is suitably sized and shaped to receive the patient interface 20 including headgear 42. Each compartment 44, 46 may be formed from a cushioning material that absorbs impact and stably supports the respective component within the compartment.

The first portion 52 also provides an elongated opening 58 that allows access to the compartment 44 and the flow generator 14 supported therein. Specifically, the side wall of the first portion 52 includes an opening 58 that exposes the front side wall 34 of the flow generator 14 when it is supported within the compartment 44. This allows access to the control panel 28 and the outlet 26 so that the user can operate and/or adjust the flow generator 14 when the case 22 is in its closed position. The opening 58 also allows for the venting/cooling of the flow generator, and for the introduction of fresh air to be pressurized by the blower of the flow generator. The bottom wall of the compartment 44 may include one or more additional openings, or be made at least partially of a porous material, to allow cooling, venting, and/or entry of fresh air to the blower inlet.

Cable Management System

As noted above, the storage case 22 provides a cable management system 50 to conveniently handle the air delivery conduit 18 and the power cord 16. The cable management system 50 allows the user to connect the air delivery conduit 18 and the power cord 16 to the flow generator 14 when the case 22 is in its open position, and then dispense and retract the connected air delivery conduit 18 and power cord 16 from the case 22 when the case 22 is in its closed position. This arrangement, along with the opening 58 in the first portion 52 that allows access to the control panel 28 of the flow generator 14, enables convenient operation of the apparatus 12 when the case 22 is in its closed position. Therefore, it is not necessary to remove the flow generator or its power cord and/or the air delivery tube from the case during operation of the flow generator in a therapy session.

In the illustrated embodiment, the cable management system 50 includes a first reel 60 structured to dispense and retract the air delivery conduit 18, and a second reel 62 structured to dispense and retract the power cord 16. The first and second reels 60, 62 are rotatably mounted to the second portion 54. In the illustrated embodiment, each reel 60, 62 includes a handle 64 to manually rotate the respective reel 60, 62 and thereby control the dispersal and retraction of the air delivery conduit 18 and the power cord 16. However, the reels 60, 62 may be rotated in any other suitable manner, e.g., spring, motor. For example, the reels 60, 62 may be spring loaded to retract and may be locked in any extended position.

When the flow generator 14 is mounted within the compartment 44, the front side wall 34 is adjacent the reel 60 to enable one end 38 of the air delivery conduit 18 to be coupled to the outlet 26 and the opposite end 40 to extend through an opening 66 provided in the second portion 54. Sufficient space is provided to enable the air delivery conduit 18 to connect to the outlet 26 while the flow generator 14 is within the respective storage compartment 44. Also, structure may be provided to align and maintain the air delivery conduit 18 in a position adjacent the opening 66. Further, a movable door 68 may be provided to selectively close the opening 66.

In an embodiment, the air delivery conduit 18 may be collapsible to a substantially flat condition in order to facilitate storage in a compact manner. In use, the air delivery conduit 18 would expand to allow the passage of air therethrough. For example, FIGS. 3B and 3C illustrate an air delivery conduit 18 including a spring 506 within the air passage. As illustrated, the spring 506 includes a Z-shaped cross-sectional configuration. The spring 506 is biased to maintain the air passage open in use (FIG. 3B), and the spring 506 is collapsible to allow the conduit to assume a substantially flat condition (FIG. 3C).

When the flow generator 14 is mounted within the compartment 44, the front side wall 36 is adjacent the reel 62 to enable one end 15 of the power cord 16 to be coupled to the power inlet and the opposite end 17 to extend through an opening 70 provided in the second portion 54. A recess 72 is provided to enable the power cord 16 to connect to the power inlet while the flow generator 14 is within the respective storage compartment 44. Also, structure may be provided to align and maintain the power cord 16 in a position adjacent the opening 70. Further, a movable door 74 may be provided to selectively close the opening 70.

Also, a cover may be provided to enclose the reels 60, 62 within the second portion 54 of the case 22. The cover may include openings to allow access to the reel handles 64 and to allow access to respective ends of the air delivery conduit 18 and the power cord 16.

When the flow generator 14 is mounted within its respective compartment 44 of the case 22, respective ends of the air delivery conduit 18 and the power cord 16 may be coupled to the flow generator 14 as shown in FIG. 2. Alternatively, the air delivery conduit 18 and the power cord 16 may be coupled to the flow generator 14 just prior to use.

When it is desired to operate the apparatus 14, the case 22 is moved into its open position to remove the patient interface 20 from its respective compartment 46, and then the case 22 is moved back into its closed position. The air delivery conduit 18 and the power cord 16 are dispensed from the respective openings 66, 70 in the case 22 by pulling on respective ends until desired lengths are achieved. Alternatively, the air delivery conduit 18 and the power cord 16 may be dispensed by rotating respective handles 64 when the case 22 is in its open position. Once dispensed, the end 40 of the air delivery conduit 18 is coupled to the patient interface 20, and the end 17 of the power cord 16 is coupled to a power outlet, as shown in FIG. 3. The flow generator 14 can then be operated while inside the case 22 via the control panel 28 that is accessible through the opening 58 in the case 22.

When the treatment session is complete, the air delivery conduit 18 is disconnected from the patient interface 20, and the power cord 16 is disconnected from the power outlet. The case 22 is moved to its open position, and the air delivery conduit 18 and the power cord 16 can be reeled into the case 22 by respective handles 64. Also, the patient interface 20 can be placed into its respective compartment 46 within the case 22. Then, the case 22 may be moved into its closed position that allows convenient transport and storage of the apparatus 12.

In illustrated embodiment, the opening 58 allows access to the control panel 28 when the case 22 is in the closed position. It should be understood that the shape, size, and/or placement of the opening 58 may vary depending on the configuration of the flow generator 14. That is, any suitable opening 58 may be provided in any suitable side wall of the case 22 to allow access to the control panel 28. Also, the opening 58 may be covered by a flexible, clear membrane, e.g., polyester film, to allow the control panel 28 to be adjusted while maintaining a closed compartment, e.g., to prevent dust ingress.

Also, it is noted that the cable management system 50 is optional and additional compartments may be provided within the case 22 to store the air delivery conduit 18 and the power cord 16. Moreover, additional openings may be provided through the case 22 to allow access to the power inlet and the outlet 26 of the flow generator 14 when the case 22 is in the closed position. The openings may take various forms, and may be configured to correspond with the shape and/or size of the power inlet and/or outlet 16. The openings would allow the air delivery conduit 18 and the power cord 16 to connect to the flow generator 14 while it is within the case 22, thereby allowing the apparatus 12 to operate while the flow generator 14 is within the case 22.

Further, the first portion 52 and/or the second portion 54 may be constructed at least in part of a sound proofing material adapted to muffle sound generated from the flow generator 14 while in use.

Storage Nest

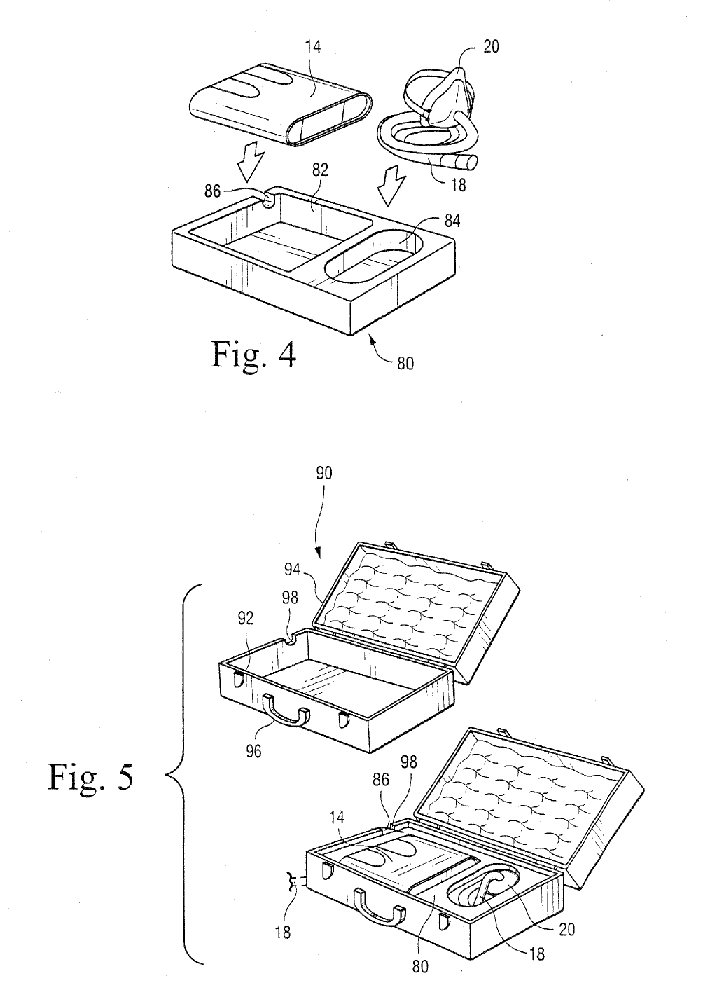

As shown in FIG. 4, the container may be in the form of a nest 80 that provides storage compartments 82, 84 adapted to conveniently store the flow generator 14, the patient interface 20, and the air delivery conduit 18. Specifically, the nest includes a bottom wall and side walls extending from the bottom wall that define storage compartments 82, 84 having upwardly facing openings adapted to receive the flow generator 14, the patient interface 20, and the air delivery conduit 18. The shape and size of each storage compartment 82, 84 generally corresponds with the shape and size of the component to be received therein. For example, the compartment 82 is suitably sized and shaped to receive the flow generator 14, and the compartment 84 is suitably sized and shaped to receive the patient interface 20 and air delivery conduit 18. Each compartment 82, 84 may be formed from a cushioning material, e.g., foam material, that absorbs impact and stably supports the respective component within the compartment. The material that surrounds the flow generator may be configured (e.g., mesh-like or porous) to allow fresh gas to enter the blower. Also, the material may provide further utility, e.g., sound proofing.

At least one of the side walls of the nest 80 provides an opening 86 that allows the air delivery conduit 18 to pass therethrough when connected to the flow generator 14. Specifically, the nest 80 includes an opening 86 that aligns with the outlet 26 of the flow generator 14 so that the user can access the outlet 26 and connect the air delivery conduit 18 thereto when the flow generator 14 is within the nest 80. The opening 86 may also be utilized to allow a power cord to connect to the power inlet of the flow generator 14. Alternatively, an additional opening may be provided through the nest 80 to allow access to the power inlet. Moreover, an opening may be provided to allow access to the control panel of the flow generator 14.

The nest 80 may be suitably sized to allow it to be removably mounted into a container for storage and/or transportation purposes. For example, FIG. 5 illustrates the nest 80 mounted within a briefcase 90. As illustrated, the briefcase 90 includes a first portion 92 movably mounted to a second portion 94, e.g., by hinges, for movement between an open position and a closed position. In the open position shown in FIG. 5, the nest 80 may be mounted into and removed from the storage space provided by the first portion 92. In the closed position, the briefcase 90 encloses the nest 80 to prevent removal therefrom. The briefcase 90 also provides a handle 96 that allows one to conveniently handle and transport the briefcase 90 when in the closed position. Additionally, the second portion 94 may provide sound proofing material adapted to muffle sound generated from the flow generator 14 while in use.

The first portion 92 of the briefcase 90 also provides an opening 98 that aligns with the opening 86 provided on the nest 80. This arrangement allows access to the outlet 26 so that the user can operate the flow generator 14 when the briefcase 90 is in the closed position. That is, the air delivery conduit 18 (in dashed lines) can extend from the outlet of the flow generator 14 and through the aligned openings 86, 98.

In an alternative embodiment, the nest 80 may be mounted into a case similar to that shown in FIGS. 1-3. Specifically, the case 22 may be modified to include a storage space in the first portion 52 adapted to receive the nest 80.

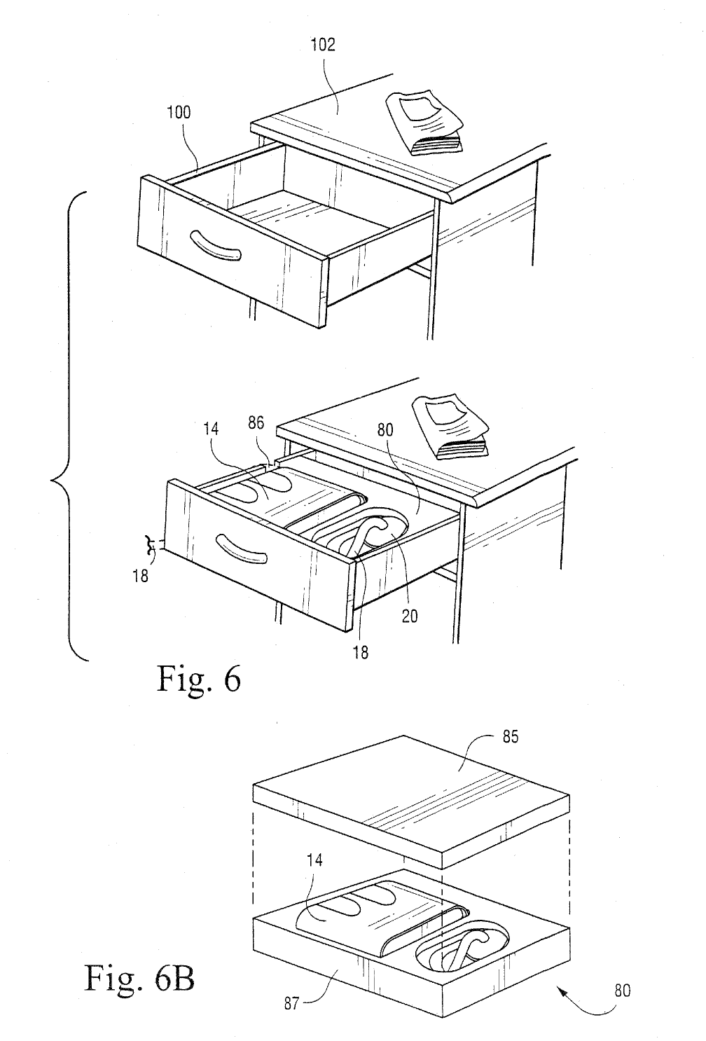

As shown in FIG. 6, the nest 80 may be mounted within the drawer 100 of a cabinet 102, e.g., bedside cabinet. As illustrated, the drawer 100 is slidably mounted to the cabinet 102 for movement between an open position and a closed position. In the open position shown in FIG. 6, the nest 80 may be mounted into and removed from the storage space provided by the drawer 100. In the closed position, the cabinet 102 encloses the nest 80 to prevent removal therefrom. The drawer 100 and/or cabinet 102 may provide sound proofing material adapted to muffle sound generated from the flow generator 14 while in use. In an embodiment, the nest 80 may include top and bottom portions 85, 87 that fully enclose the flow generator 14 with a cushioning material, e.g., foam, to provide optimal sound proofing as shown in FIG. 6B.

The drawer 100 also provides an opening that aligns with the opening 86 provided on the nest 80. This arrangement allows access to the outlet 26 so that the user can operate the flow generator 14 when the drawer 100 is in the closed position. That is, the air delivery conduit 18 (in dashed lines) can extend from the outlet of the flow generator 14 and through the aligned openings.

It is noted that the nest 80 may be mounted in any other suitable storage space. Moreover, the nest 80 may be suitably modified to include storage compartments of various shapes and sizes, and one or more openings to allow access to the compartments.

Slim Flow Generator Design

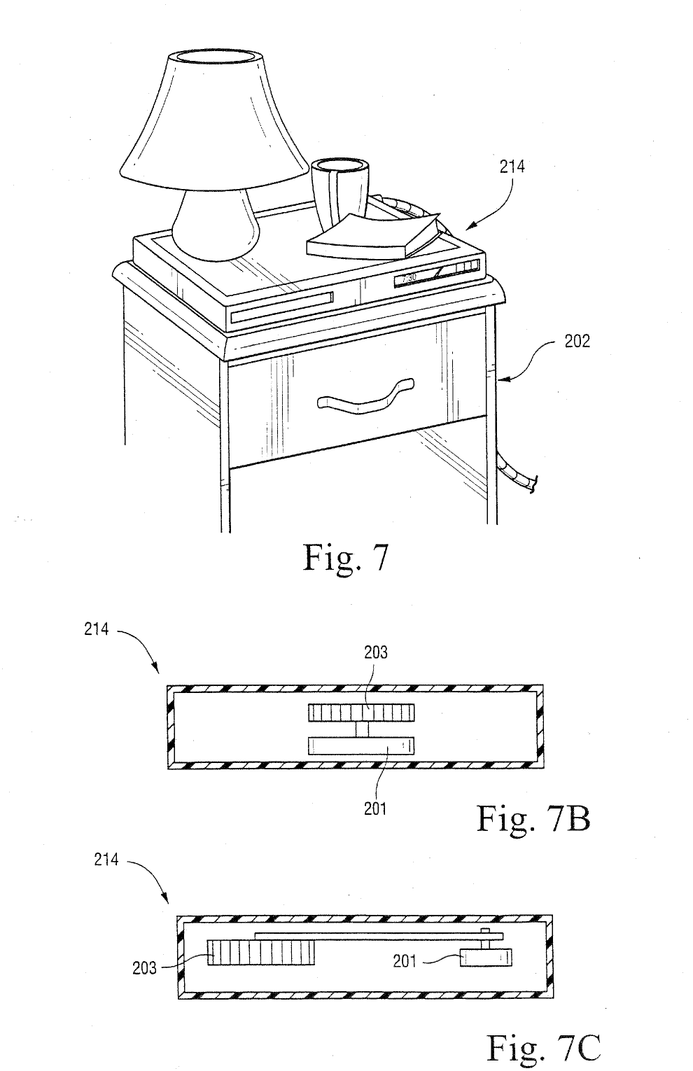

The flow generator itself may be structured to facilitate transport and convenient storage. For example, FIGS. 7-10 illustrate a flow generator 214 having a relatively slim streamline design (similar to the flow generator 14 illustrated above). Specifically, the flow generator 214 is structured such that the housing walls have substantially flat configurations that provide substantially flat exterior surfaces. Moreover, the flow generator 214 is structured such that the length and width are substantially longer than the height. Thus, the height is relatively short, thereby providing a thin configuration. This configuration allows the flow generator 214 to be placed in tight locations, thereby concealing its location while still making it convenient for use.

In an embodiment, the motor of the flow generator 214 may have a substantially flat configuration to enable the “slim” flow generator design. An example of such a flat motor is disclosed in “Engineering Penn State”, The Magazine of the Penn State College of Engineering, Summer 2003, Vol. 19, No. 3. As shown in FIG. 7B, the blower 203 may be axially aligned with the flat motor 201. Alternatively, as shown in FIG. 7C, the blower 203 may be offset from the flat motor 201, e.g., side by side. Ideally, the motor/blower configuration is thin enough to prevent mattress bulge (e.g., see FIG. 9).

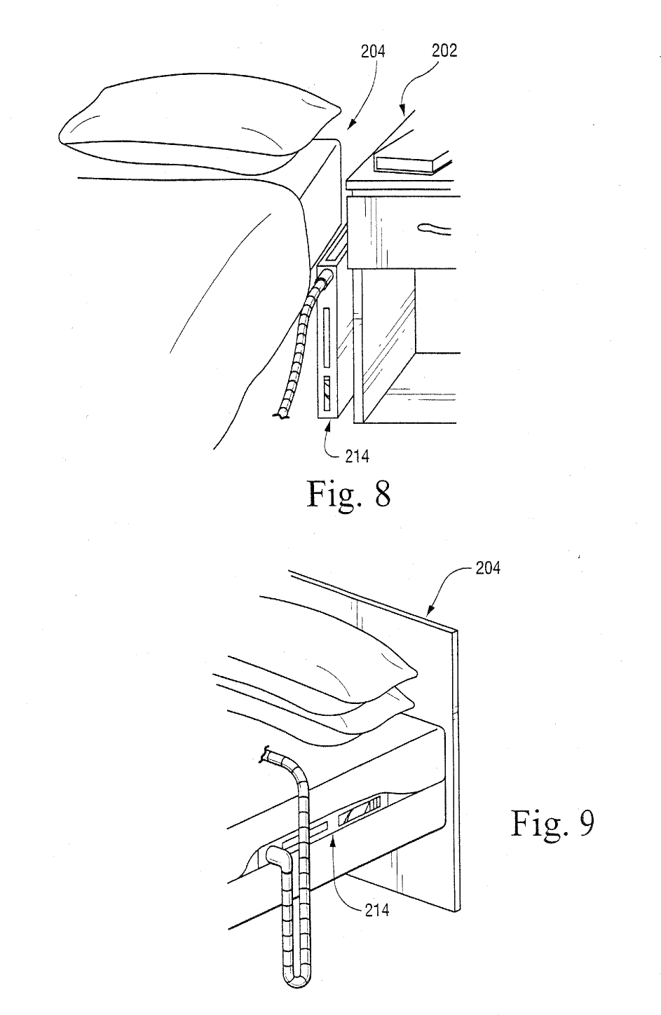

As shown in FIG. 7, the flow generator 214 may be supported on a bedside table 202 in a horizontal orientation. As noted above, the upper wall of the housing is substantially flat, which allows it to support one or more objects (e.g., glass, book, lamp, etc.) thereon in a stable manner. As shown in FIG. 8, the slim flow generator design allows the flow generator 214 to positioned in a vertical orientation in the limited space between a bed 204 and a bedside table 202. Also, the slim design allows the flow generator 214 to positioned in a horizontal orientation under the mattress of a bed 204 as shown in FIG. 9.

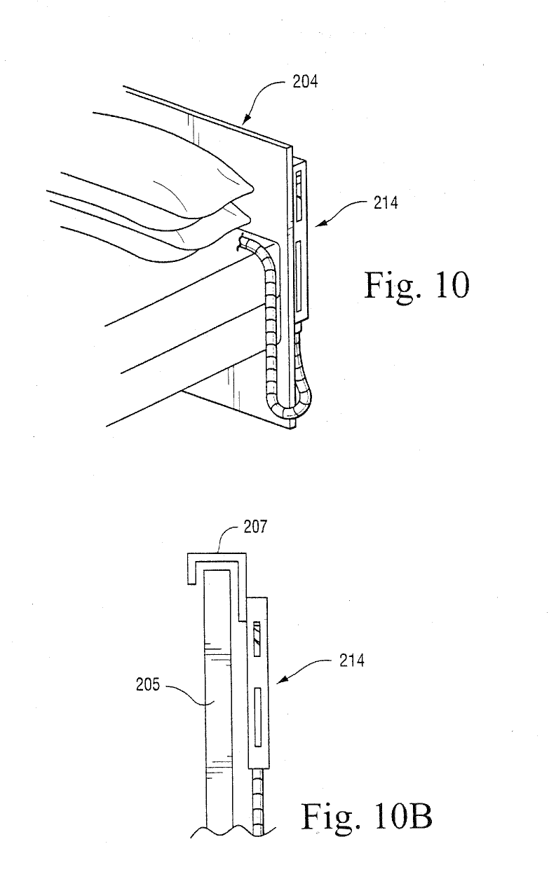

In another embodiment, the flow generator 214 may include structure, e.g., brackets, that allows it to be mounted. For example, as shown in FIG. 10, the flow generator 214 may be mounted behind a bed 204, e.g., to headboard of the bed or to the wall adjacent the bed. In an embodiment, the bracket may be attached to the bed 204 and the flow generator 214 may be removably attached to the bracket, e.g., clips on/off the bracket. As shown in FIG. 10B, a bracket 207 may be attached to the flow generator 214, e.g., removably attached, that is adapted to engage the headboard 205 of the bed 204.

It is noted that the control panel, outlet, and power inlet of the flow generator 214 may be provided on any suitable wall of the housing. Moreover, the location of these features may be determined at least in part on the desired storage location of the flow generator.

Flow Generator Stand

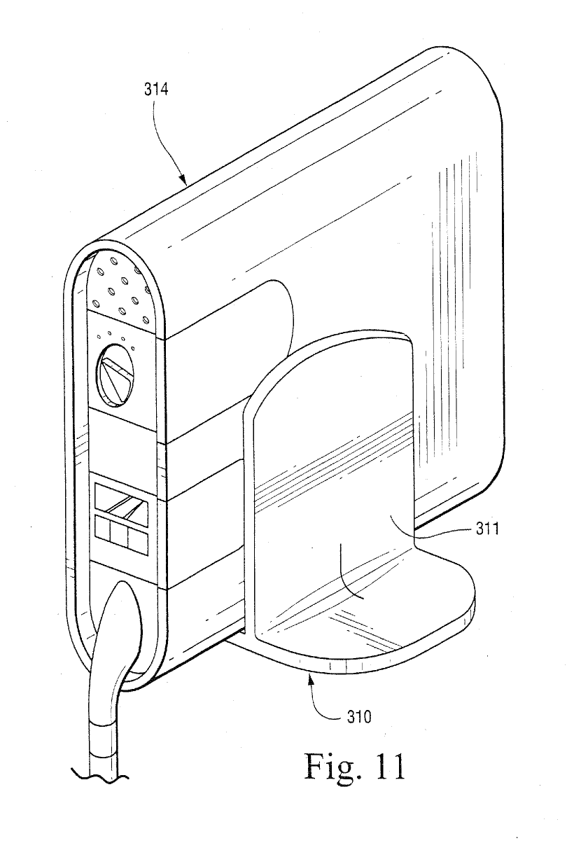

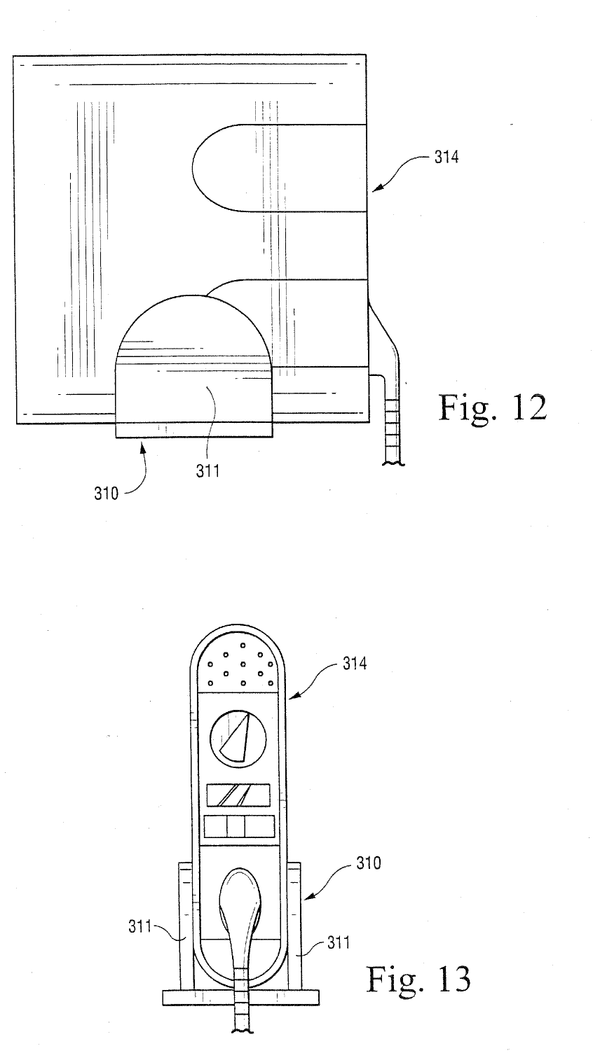

As shown in FIGS. 11-13, a stand 310 may be provided along with a flow generator 314 to support the flow generator 314 in a generally vertical orientation. Thus, the stand 310 constitutes a storage system adapted to conveniently store the flow generator 314. In the illustrated example, the flow generator 314 has a relatively slim design (e.g., similar to the flow generators 14, 214), and the stand 310 includes spaced apart arms 311 that engage opposing walls of the flow generator 314 to stably support the flow generator 314 in the vertical orientation. This arrangement allows the flow generator 314 to be conveniently and stably positioned in any suitable location. One or both of the arms can be adjustable (e.g., via a sliding track arrangement or a pin and groove arrangement or other conventional adjustment assemblies) to allow the support of flow generators having various widths. For example, the stand 310 may accommodate flow generators having a width of between 2-20 cm.

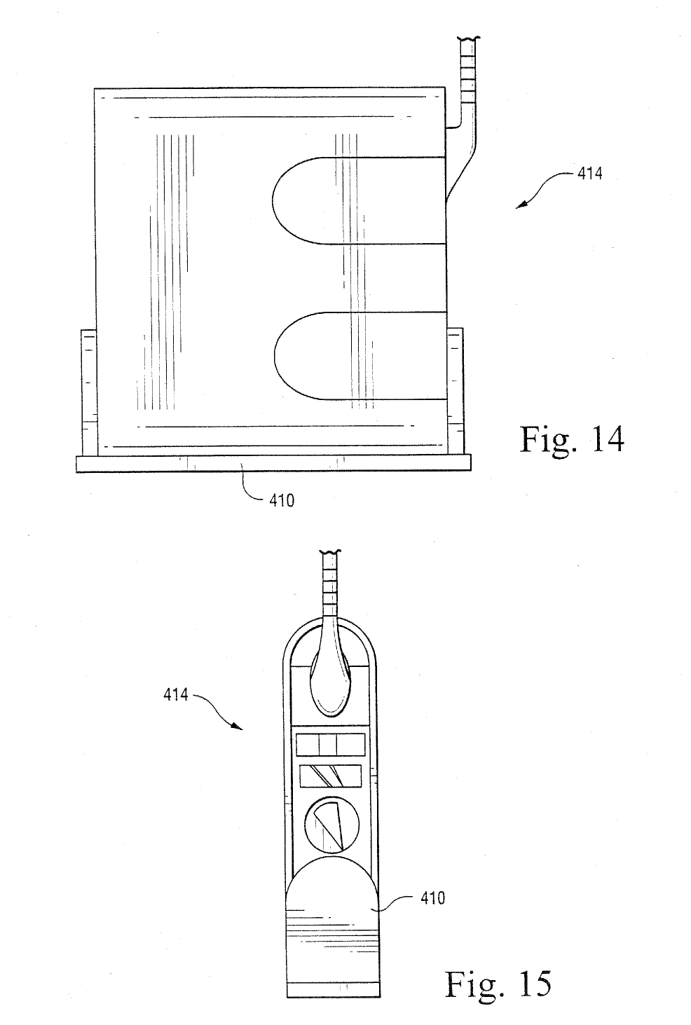

In another embodiment, a stand 410 may be structured to support the flow generator 414 across its length rather than its width, as shown in FIGS. 14 and 15. Similar to the above, one or both of the arms of the stand 410 may be adjustable to allow the support of flow generators having various lengths. The stand and flow generator may include cooperating structure, e.g., a tongue and groove arrangement, to prevent the flow generator from rotating relative to the stand, thereby holding the flow generator upright.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. In addition, while the invention has particular application to patients who suffer from OSA, it is to be appreciated that patients who suffer from other illnesses (e.g., congestive heart failure, diabetes, morbid obesity, stroke, barriatric surgery, etc.) can derive benefit from the above teachings. Moreover, the above teachings have applicability to storage of components in general for use with patients and non-patients alike.

What is claimed is:

1. A flow generator assembly, comprising:
a flow generator structured to generate a supply of pressurized breathable air to a patient; and
a bracket attachable to the flow generator and adapted to mount the flow generator to a bed or a wall adjacent the bed.

2. A flow generator assembly according to claim 1, wherein the bracket is adapted to engage a headboard of the bed.

3. A flow generator assembly according to claim 1, wherein the bracket is removably attached to the flow generator.

4. A flow generator assembly according to claim 3, wherein the flow generator clips on and off the bracket.

5. A flow generator assembly according to claim 1, wherein the flow generator comprises a height, a width, and a length, and the length and/or the width are greater than the height.

6. A flow generator according to claim 5, wherein the flow generator comprises a housing having at least six flat housing walls.

7. A flow generator assembly according to claim 6, wherein the flow generator comprises a flat motor and a blower.

8. A flow generator assembly according to claim 7, wherein the flat motor and the blower are axially aligned.

9. A flow generator assembly according to claim 7, wherein the flat motor and the blower are offset.

10. A flow generator assembly according to claim 2, wherein the bracket comprises a first member, a second member substantially perpendicular to the first member, and a third member substantially perpendicular to the second member and substantially parallel to the first member.

11. A flow generator assembly according to claim 10, wherein the first member is longer than the third member.

12. A flow generator assembly according to claim 10, wherein the second member is longer than the third member.

13. A flow generator assembly according to claim 11, wherein the first member is attachable to the flow generator.

14. A flow generator assembly according to claim 13, wherein the bracket is between the flow generator and the headboard when the bracket engages the headboard.

15. A flow generator assembly according to claim 1, wherein the flow generator comprises a control panel, an outlet attachable to an air delivery conduit, and a power inlet attachable to a power cord.

16. A flow generator assembly according to claim 15, wherein the outlet is provided on a side of the flow generator opposite a side of the flow generator attached to the bracket.

17. A flow generator assembly according to claim 16, wherein the outlet side of the flow generator is substantially perpendicular to a side of the flow generator comprising the control panel.

18. A flow generator assembly according to claim 16, wherein the outlet side of the flow generator also comprises the control panel.
